(19) United States
(12) Patent Application Publication
(10) Pub. No.: US 2002/0127715 A1
(43) Pub. Date: Sep. 12, 2002

(54) Transfection of human embryonic stem cells

(76) Inventors: Nissim Benvenisty, Jerusalem (IL); Ofra Yanuka, Givat Zeev (IL); Maya Schuldiner, Moshav Aminadav (IL); Rachel Eiges-Avner, Mevasseret Zion (IL)

Correspondence Address: BROMBERG & SUNSTEIN LLP, 125 SUMMER STREET, BOSTON, MA, 02110-1618, US

(21) Appl. No.: 09/995,452

(22) Filed: Nov. 27, 2001

Related U.S. Application Data
(60) Provisional application No. 60/253,222, filed on Nov. 27, 2000.
(60) Provisional application No. 60/267,664, filed on Feb. 9, 2001.

(51) Int. Cl. C12N 5/08; C12N 15/87

(52) U.S. Cl. 435/366; 435/455

(57) ABSTRACT

Methods are provided for altering gene expression in a population of human embryonic cells that include introducing a gene expression altering sequence into cells the cells retaining their pluripotent character, for purifying pluripotent embryonic stem cells from a heterogeneous population of cells, and for treating a human suffering from a deficiency of a selected cell type. Reagent cell populations are further provided for supplying material for transplantation consisting essentially of pluripotent human embryonic stem cells modified by foreign genetic material.

### CROSS REFERENCE TO RELATED APPLICATIONS

This application gains priority from provisional application 60/253,222, filed Nov. 27, 2000 and 60/267,664 filed Feb. 9, 2001, these applications being incorporated by reference herein.

### TECHNICAL FIELD AND BACKGROUND ART

The present invention relates to preparations and methods of transfecting human embryonic stem cells, forming clonal preparations of pluripotent stem cells and enhancing a cell population in a human subject.

It is known in the prior art that embryonic stem (ES) cells are pluripotent cells derived from the inner cell mass (ICM) of an in vitro fertilized embryo grown to the blastocyst stage. These cells are unique in their ability to grow indefinitely in culture while retaining a normal karyotype. Embryonic stem cells were first isolated from mice and were found to form aggregates or embryoid bodies in vitro which spontaneously differentiated into various cell types (Robertson, (1987) in Teratocarcinomas and Embryonic Stem cell, A Practical Approach, pp. 71-112). More recently, it has been shown that human embryonic stem cells can differentiate in culture to cells of the three germ layers that arise in initial stages of embryonic development (Itskovitz-Eldor, J. et al., (2000) Mol. Med., Vol. 6, pp. 88-95) and that their differentiation potential may be manipulated by the use of growth factors (Schuldiner, et al., (2000) Proc. Natl. Acad. Sci. U.S.A.). Various cell types may be identified in the differentiated human ES cells, e.g. neurons (Schuldiner, M., (2001) Brain res.), pancreatic β cells (Assady, S. et al., (2001) Diabetes, Vol.50, pp. 1691-1697), cardiomyocytes (Kehat, I. et al. J. Clin. Invest. 108, 407-414. (2001), and hematopoietic cells (Kaufman et al., (2001) Proc. Natl. Acad. Sci. U.S.A., Vol. 98, pp. 10716-10721).

Embryonic stem cells (ES) provide a potentially unlimited supply of differentiated cells. The properties of stem cells that include self-renewal and vast differentiation potential are of major importance in such procedures as they serve as a solution for the overall lack of tissue sources. Studies in the mouse, rat and non-human primates have shown that transplantations of embryonic and adult stem cells into tissues such as pancreas (Ramiya et al., (2000) Nat. Med, Vol. 6, pp. 278-282), brain (Isaacson et al., (1989) Exp. Brain Res., Vol. 75, pp. 213-220, Lee et al., (2000) Brain, Vol. 123, pp. 1365-1379, Deacon et al., (1998) Exp. Neurol., Vol. 149, pp. 28-41), spinal cord (McDonald et al., (1999) Nat. Med., Vol. 5, pp. 1410-1412), heart (Klug et al., (1996) J. Clin. Invest., Vol. 98, pp. 216-224) and bone marrow (Gutierrez-Ramos et al., (1992) PNAS, Vol. 92, pp. 9171-9175, Palacios et al., (1995) PNAS, Vol. 92, pp. 7530-7534) have to some extent resulted in penetration and normal differentiation in the host tissue followed, in some instances, by phenotypic improvement of the animal's health (McDonald et al., (1999).

Recently, human fetal brain cells were injected into the nigra-striatum of patients suffering from Parkinson's disease. The grafted cells caused improvement in most patients, though in some patients the transplantation caused serious adverse reaction probably due to uncontrollable proliferation of the fetal cells (Freed et al., (2001) N. Engl. J. Med., Vol. 344, pp. 710-719). Such deleterious results, alongside tumor formation are some of the clinical problems that may arise through the use of non-terminally differentiated cells that could possibly still proliferate in the host tissue. Safety steps are needed to deactivate or destroy cells that prove to be harmful to the patient.

In particular, human ES cells may be uniquely useful as an unlimited source of cells for the medical procedure of transplantation in numerous pathologies, and as a component in biomedical engineering as well as providing clues on early stages of human development. However, the clinical problems associated with transplantation and biomedical engineering should be addressed including problems associated with the reaction to the foreign cells by the host immune system, residual proliferation of the cells after transplantation and tumor formation or ectopic and excessive differentiation.

Genetic manipulation of differentiation of human ES cells would be desirable so as to obtain a uniform population of precursors or fully differentiated cells for in vivo or in vitro use.

### SUMMARY OF THE INVENTION

In a first embodiment of the invention there is provided a method of altering gene expression in a population of human embryonic stem cells that includes: introducing a polynucleotide into the population of cells, the polynucleotide containing a gene expression altering sequence so that gene expression in the embryonic stem cells prior to introducing the polynucleotide is measurably different from gene expression after introducing the polynucleotide. In an example of the method the expression altering sequence is an enhancer for modulating gene expression in the population of embryonic stem cells. The expression altering sequence may be a gene encoding a protein, the protein not normally being expressed in the population of embryonic stem cells and the protein may be selected from a fluorescent protein and an antibiotic resistance protein. The fluorescent protein may be for example any of green fluorescent protein, lacZ, firefly Rennila protein, luciferase, red cyan protein and yellow cyan protein. The antibiotic resistance protein may be for example any of hygromycin, neomycin, zeocin and puromycin.

According to the above, introducing polynucleotides into cells may be facilitated by formulations that include a cationic lipid reagent, cationic non-lipid polymer transfection reagent, a liposomal transfection reagent for introducing into the population of cells. Alternatively, electroporation may be used.

In another embodiment of the invention, a method is provided for altering gene expression in a population of human embryonic stem cells; that includes: introducing into the population of cells by electroporation or in the presence of a cationic polymer, a DNA sequence corresponding to at least one of an enhancer, a promoter and a gene so as to alter gene expression in the population of embryonic cells in an amount to permit cells containing the DNA sequence to be distinguished from cells absent the DNA sequence.

According to this embodiment, the gene may encode a protein selected from a fluorescent protein, a suicide gene, a knockout protein and an antibiotic resistance protein. The promoter may be selected from rex-1, oct-4, oct-6, SSEA-3, SSEA-4, TRA1-60, TR1-81, GCTM-2, alkaline phosphatase, and Hes1 promoters, the fluorescent protein may be selected from green fluorescent protein, lacZ, firefly Rennila protein, luciferase, red cyan protein and yellow cyan protein, the antibiotic resistance protein may be selected from hygromycin, neomycin, zeocin and puromycin, the suicide gene may be an inducible apoptic gene or encode a protein selected from herpes simplex thymidine kinase, inducible Diptheria toxin, bacterial cytosine deaminase, and the knockout may causes the DNA sequence to be inserted into and render non-functional a gene encoding beta 2 microglobulin, HLA-1, HLA-2 or an INF receptor.

In another embodiment of the invention, a method is provided for purifying pluripotent embryonic stem cells from a heterogeneous population of cells, comprising: introducing into the cells, a DNA encoding a selectable marker under a promoter that is specifically active in undifferentiated cell; separating those cells expressing the selectable marker from cells not expressing the marker; and obtaining purified pluripotent cells. Accordingly, the selectable marker may be a fluorescent marker for example, green fluorescent protein, lacZ, firefly Rennila protein, luciferase, red cyan protein or yellow cyan protein. The cells containing the marker may be seperated from the cells lacking the marker using a fluorescent activated cell sorter or a laser scanning cytometer. Where the selectable marker is an antibiotic resistance marker, separating cells expressing the marker from those that cannot can be achieved by culturing the cells in a selective medium containing antibiotic.

In an embodiment of the invention, a method is provided for treating a human subject for a condition resulting from a deficiency of a selected cell type, that includes causing human embryonic stem cells to be transfected in vitro with a nucleic acid containing a marker under a tissue specific promoter; separating the selected cell type expressing the marker from cells not expressing the marker, and introducing the selected cell type into the subject so as to treat the condition. The nucleic acid may further contain a suicide gene. The suicide gene may be an inducible apoptic gene or encodes a protein selected from herpes simplex thymidine kinase, inducible Diptheria toxin, bacterial cytosine deaminase. The marker may be a fluorescent marker or an antibiotic resistance protein. The fluorescent protein may be any of green fluorescent protein, lacZ, firefly Rennila protein, luciferase, red cyan protein and yellow cyan protein. The antibiotic resistance protein may be any of hygromycin, neomycin, zeocin and puromycin. The cells may be transfected by means of a cationic polymer transfection reagent including lipophilic or nonlipophilic reagents and liposomal reagents or by electroporation. In embodiments of the method, the nucleic acid may not contain viral genes. The selected cell type may be one of epidermal cells, dermal cells, muscle cells, cartilage cells, osteoblasts, osteoclasts, neurons, retinal cells, endodermal cells, hematopoietic cells, cells of the immune system or may be specialized cells from any functionally distinct organ in the human subject. The cell type may be administered to the subject by injection. Examples of conditions for treating with a selected cell type includes cancer, immune disorders, autoimmune diseases, diseases of aging, degenerative diseases including neurodegenerative diseases, and conditions associated with trauma.

In an embodiment of the invention, a cell population is provided that includes a substantially pure population of human embryonic stem cells containing an expression altering sequence of exogenous DNA.

In a further embodiment of the invention, a method is provided of producing a clonal pluripotent cell population from a mixture of pluripotent and differentiated cells; including: transfecting the mixture of cells in the presence of a cationic polymer or by electroporation with a DNA encoding a marker protein under a promoter that is selectively active in cells of the inner cell mass of the embryo; and separating the embryonic stem cells from the differentiated cells according to the presence or absence of an expressed marker so as to produce the clonal pluripotent cell population. The promoter may be selected from rex-1, oct-4, oct-6, SSEA-3, SSEA-4, TRA1-60, TR1-81, GCTM-2, alkaline phosphatase, and Hes1 promoters. DNA transfection may occur in the presence of a cationic polymer. The marker protein may be selected from a fluorescent protein and an antibiotic resistance protein. The fluorescent protein may be selected from green fluorescent protein, lacZ, firefly Rennila protein, luciferase, red cyan protein and yellow cyan protein, the antibiotic resistance protein may be selected from hygromycin, neomycin, zeocin and puromycin.

An embodiment of the invention includes a method for regulating cell viability of a population of cells in a subject, wherein the population of cells are derived from a human embryonic stem cell culture which has undergone directed differentiation, the method including introducing the population of cells into the subject, the population of cells containing an exogenous DNA encoding a suicide sequence, wherein the population of cells are selected from the group consisting of: undifferentiated cells, partially differentiated or wholly differentiated cells; and treating the subject with an agent for activating the suicide sequence in the cells if the cells give rise to adverse events in the subject so as to cause cell death of the cells. For example, the dverse events may be hyperproliferation of the introduced cells.

In an embodiment of the invention, a method is provided for screening an agent to determine an effect on differentiation of cells in vitro, comprising: adding the agent to an in vitro cell culture of a population of genetically engineered human embryonic stem cells expressing a detectable marker under a cell specific promoter; providing the conditions for the embryonic stem cells to differentiate; and determining the effect on differentiation of the agent. The detectable marker may be a fluorescent marker or an antibiotic resistant marker.

In an additional embodiment of the invention, a reagent cell population for supplying material for transplantation is provided consisting essentially of pluripotent human embryonic stem cells modified by foreign genetic material which is DNA not normally present in embryonic stem cells; which occurs in embryonic stem cells but is not expressed in them at levels which are biologically significant; DNA which occurs in embryonic stem cells and has been modified so that it is only expressed by selected derivative cells; or any DNA that can be modified to be expressed by embryonic cells, derivative cells alone or in any combination thereof. For example, the foreign genetic material comprises genetic material encoding at least one selectable marker. At least one selectable marker may be a dominant selectable marker for example a gene encoding antibiotic resistance, a gene encoding a suicide protein, a fluorescent protein or an antibiotic resistant protein. The suicide gene may be hsv-tk and the suicide protein thymidine kinase, the fluorescent protein may be green fluorescent protein, lacZ, firefly Rennila protein, luciferase, red cyan protein or yellow cyan protein and the antibiotic resistance protein, hygromycin, neomycin, zeocin or puromycin.

### BRIEF DESCRIPTION OF THE DRAWINGS

The foregoing features of embodiments of the invention will be more readily understood by reference to the following detailed description, taken with reference to the accompanying drawings, in which:

FIG. 1 shows the efficiency of different techniques to introduce DNA into human embryonic stem cells. The scale bar is 100 μm. Inset :human ES cells transiently transfected with EGFP under the control of the housekeeping gene E1F (elongation factor 1) promoter. Green fluorescent ES cellss incorporate the foreign DNA.

FIG. 2 shows transfection of human embryonic stem cells (a) transient transfection; and (b) stable transfection.

FIG. 3 shows electroporation of human embryonic stem cells.

FIG. 4 shows the expression of a transfected marker of undifferentiated cells as a method to identify and isolate pluripotent human embryonic stem cells. (a) Human ES cells underwent stable transfection with EGFP fused to murine Rex 1 minimal promoter sequence. The transfected ES cells and their differentiated cell derivatives are shown: simple embryoid body (sEB) and mature embryoid body (maEBs). The left and middle columns are photos of bright bright and dark fields respectively. The right column is the overlay of the two photos. Note that only the undifferentiated cells are fluorescent. The fluorescent ES colony is surrounded by differentiated non fluorescent cells. The simple EB is labeled only in the middle and nt in the peripheral primitive endodermal cells. Mature EBs are generally not fluorescent and only very distinct areas in them are fluorescent (probably residual fluorescent cells). The scale bar indicates 100 μm. (b) The stable transfection of human ES cells with constitutively expressed EGFP construct, driven by mouse PGK promoter. Overlay photos of the dark on bright field of the transfected ES cells (ES) and their differentiated cell derivatives are shown:simple embryoid bodies (sEB), mature embryoid bodies (mEB) and differentiating embryonic cells derived from dissociated embryoid bodies (DE). Note that GFP is expressed by all cells differentiated and undifferentiated in theproliferating ES colony as well as by all cells of simple and mature Ebs (including those in the outer layer of the sEB, where differentiation of primitive endoderm is taking place in the mouse EB. The scale bar is 100 μm.

FIG. 5(A) shows FACS analysis of Human ES cell transfected with Rex1-EGFP construct according to their intensity of green fluorescence emission. Cell samples of MEF (feeder layer) and undifferentiated human ES (grown in the presence of feeder cells) were used as controls. Fluorescent intensity was then compared between undifferentiated human ES cells, transfected human ES cells and their differentiated cell culture derivatives (obtained by growth in non-supplemented gelatin-coated plates). (A-1) MEF, (A-2) undifferentiated ES cells and MEF, (A-3) undifferentiated transfected ES cells plus MEF; and (A-4) partially differentiated transfected ES cells on gelatin.

FIG. 5(B) shows cells sorting of three GFP-expressing cell lines performed by FACS. Following trypsin digestion, cell samples (3-4 replicates per clone) were evaluated for percentage of cell viability (84%) and sorted according to intensity of green fluorescent emission. The collected cell samples (25,000-50,000) were redetermined for cell viability (86%) and replated on MEF^{neo+}culture dishes (2,500-8,000 cells per dish). Following growth in vitro, cell culture dishes were inspected and recorded for total number of proliferating human ES cells (20%±4% [n=11].

FIG. 5(C) shows photos of fluorescent labeled proliferating human ES colonies (top, bright field and bottom, dark field)obtained 4 days after cell sorting by FACS.

FIG. 6 shows the effects of Ganciclovir on human ES Cells expressing the herpes simplex thymidine kinase gene. (A) Human ES cell clones expressing the HSV-TK gene showing sensitivity to the presence of the pro-drug Ganciclovir (Ganc) (B) Dose response of HSV-TK+ clones to Ganciclovir treatment. Six clones (TK+) and human ES cells as controls were grown in presence of various concentrations of Ganciclovir. The bars represent Standard error values (C) Time course of the effects of Ganciclovir on HSV-TK+ cells. Six HSV-TK+ clones and control human ES cells were treated with 2×10⁻⁶M Ganciclovir for 9 days. The experiment was repeated 4 times. After 9 days Ganciclovir was removed from the media and cells were grown for additional 10 days. The bars represent Standard error values.

FIG. 7 shows selective ablation of HSV-TK+ cells (A) Analysis of mixed cultures of GFP expressing cells (GFP+) with HSV-TK+ (TK+) cells. Following treatment with Ganciclovir selective elimination of the HSV-TK+ cells is shown by FACS analysis of GFP expression.

FIG. 8 shows sensitivity of teratoma cells to Ganciclovir (A) Differentiated HSV-TK+ teratoma cells showing sensitivity to the presence of the pro-drug Ganciclovir (Ganc.) (B) Time course of differentiated HSV-TK+ teratoma cells in response to 2×10⁻⁶ M Ganciclovir during 8 days. After 8 days Ganciclovir was removed from the media and cells were grown for additional 8 days. The bars represent Standard error values.

FIG. 9 Retention of normal karyotype and differentiation potential by the hsTK+ cell lines (A) Karyotype of an HSV-TK+ clone using G-banding. All cell lines have a normal, XX chromosome set (B) RT-PCR analysis of differentiation markers from the three germ layers on human ES cells, hsTK+ embryoid body (EB) and teratoma (Ter) cells. The markers used were: α-fetoprotein (αFP), albumin, amylase, β-globin, cardiac actin (cActin), enolase, keratin, glial fibrillary acidic protein (GFAP) and neurofilament heavy chain (NF-H). As control for presence of cDNA the housekeeping genes β-actin and glyceraldehyde-3-phosphate hydrogenase were used.

### DETAILED DESCRIPTION OF SPECIFIC EMBODIMENTS

Definitions. As used in this description and the accompanying claims, the following terms shall have the meanings indicated, unless the context otherwise requires:

“Transfection” is the introduction of nucleic acid into cells in a population. Transfection may occur in vivo as well as in vitro. One result of transfection is to produce a genetically engineered cell.

“Vector” is any commercial vector such is available in the market, for example, vectors from Clontech, Promega, InVitrogen or New England Biolabs.

“Suicide sequence” in a cell is any DNA which when activated as a result of an externally administered agent acting either directly on the DNA, RNA or on protein expressed by the DNA results in apoptosis or damage to the cells containing the suicide sequence. Suicide genes can be under a constitutive promoter or a tissue specific promoter for example an ES cell specific promoter. When the cells are transplanted into a subject in vivo, the externally administered agent may be provided orally or parenterally including by subcutaneous, intramuscular or intravenous injection or by transdermal means. Examples of suicide genes are inducible apoptotic genes and those encoding thymidine kinase, bacterial cytosine deaminase, inducible Diptheria toxin.

“Transcription factors” refer to any of DNA, RNA or protein that become associated with the DNA to be transcribed and modulate the extent of transcription. Examples of transcription factors include hepatic nuclear factors (such as HNF3), muscle specific factors (such as MyoD), hematopoietic factors, pancreatic factors (such as PDX-1), homeobox genes and others known in the art.

“Cell specific promoters” are generally non-coding nucleotide sequences located upstream from a gene and regulate the tissue specific expression. For example, neuronal specific promoters are located upstream from encoding sequence for neurofilament heavy chain; cardiac promoters determine expression of cardiac proteins and actins in cardiac muscle cell, hematopoietic promoters determine expression of globin proteins including beta globin, a liver promoter regulates expression of albumin in hepatocytes, and a pancreatic promoter regulates expression of insulin. Other examples of promoters are those that regulate expression of nestin, tyrosine hydroxylase, dopamine beta hydroxylase, CD34, PGX-1, albumin, ISL-1 and ngn-3. These examples are not intended to be limiting.

“Markers” are DNA, RNA or protein that can be readily detected in cells and provide a means of distinguishing those cells containing the marker from those that lack the marker. Markers can be used to track cellular events in circumstances involving a changing environment. Markers can be intrinsic in the cells of interest or may be foreign and introduced into the cells to express proteins. For example, where foreign DNA encodes markers these are sometimes called reporter genes. “Reporter genes” are those genes that “report” the presence of particular cells and may include cell specific enhancers and promoters that control whether tissue specific expression of a gene occurs and how it is modulated. Reporter genes may be introduced into cells by transfection. Transfection of cells with genes encoding reporter proteins provides a means for tracking cells. Examples of reporter genes include green fluorescent protein, Lac Z, firefly Rennila protein, red, yellow or blue cyan fluorescent proteins or other fluorescent protein, including those found in marine animals. Other markers include antibiotic resistance proteins to protect cells against for example, neomycin, hygromycin, zeocine and puromycin.

“Expression altering sequence” includes any exogenous nucleic acid which when introduced into target cells either extra-chromosomally or chromosomally, has the ability to modulate gene expression in the same cell or by association, in other cells, by either enhancing or suppressing expression of proteins already being made in the cell or by causing a protein to be expressed that would not normally be expressed but which is naturally encoded by DNA in the cell, or by causing a protein to be expressed that would not normally be expressed in the cells and is not naturally encoded by DNA in the cell such as the protein from one species in cells from another species. Examples of expression altering sequences include: enhancers, promoters, transcription activators and genes as well as sequences for enhancing recombination including homologous recombination, between proximate DNA molecules. Cells expressing particular genes in suitable quantities may be used in cell therapy in a subject to correct defective gene expression associated with a condition in the subject. Examples of therapeutically beneficial proteins expressed by genes in differentiated cells derived from human embryonic stem cells include growth factors such as epidermal growth factor, basic fibroblast growth factor, glial derived neurotrophic growth factor, nerve growth factor, insulin-like growth factor (1 and 11), neurotrophin-3, neurotrophin-4/5, ciliary neurotrophic factor, AFT-1, lymphokines, cytokines, enzymes—for example, glucose storage enzymes such as glucocerebrosidase, tyrosine hydroxylase.

“Condition” describes a state that is manifest as different from normal and for which a human subject may seek treatment. Examples of conditions include cancers such as late stage cancers including ovarian cancer and leukemia, diseases that compromise the immune system such as AIDS and autoimmune diseases such as multiple sclerosis, diabetes mellitus, inflammatory bowel diseases such as Crohn's disease, systemic lupus erythmetosus, psoriasis, rheumatoid arthritis, autoimmune thyroid disease and scleroderma, conditions affecting the nervous system such as muscular dystrophy, Alzheimer's disease, Parkinson's disease, spinal cord injuries, liver diseases such as hypercholesterolemia and other conditions for which replacement of damaged tissue is desirable such as in heart disease, cartilage replacement, burns, foot ulcers, gastrointestinal diseases, vascular diseases, kidney diseases, urinary tract disease and aging related diseases and conditions. The condition may be associated with defective genes, e.g. defective immune system genes, cystic fibrosis genes or other genetic diseases.

We have established herein methods and compositions for genetically engineering human ES cell lines and we describe an efficient protocol for transfecting these cells. By introducing genetic modifications into their genome, we can manipulate these cells in vitro, we can purify stem cell clones using selectable markers and we can use, track, manipulate and if desired, kill engineered stem cells in cell based therapies as well as for other biomedical and research purposes.

Markers can be used to isolate specific cell types from a heterogeneous culture. For example, when a population of cells is transfected with a DNA containing a gene that codes for a drug resistance and is driven by a tissue specific promoter then regardless of the cell types that contain the gene, the only cell which will survive in the presence of the drug is the cell type capable of expressing the drug resistance gene.

Human embryonic stem cells which have been genetically modified as described below may be used for many purposes not only related to learning more about the biology of differentiation but also for cell therapy in human subjects. Such uses include the following: (a) tracking the steps of differentiation of the embryonic stem cells, (b) identifying factors that affect differentiation and self renewal, (c) separating pluripotent stem cells from partially differentiated stem cells, (d) characterizing and selecting different types of differentiated cells, (e) isolating pure preparations of differentiated cells, (f) improving the yield of selected differentiated cells, (g) monitoring transplanted cells in vivo including monitoring cell movement, proliferation, and location in vivo, (h) destroying transplanted cells undergoing hyperproliferation in vivo including conversion to teratocarcinomas and (i) eliminating MHC related molecules to avoid transplant rejection.

Although ES cell lines are now available for a large array of mammalian species (including hamster, mink, pig, rabbit, sheep, cattle, common marmoset and rhesus monkey (for a review see Prelle et al., (1999) Cell Tissues Organs, Vol. 165, pp. 220-236) and for humans (U.S. application Ser. No. 09/918,702) these ES cell lines other than those of mice have not been transfected with exogenous DNA. In murine ES cells, electroporation was found to be the method of choice for introducing foreign DNA into ES cells (Thomas et al., (1987) Cell, Vol. 51, pp. 503-512). In contrast to mouse embryonic stem cells, we found that although human ES cells can be transfected by electroporation, improved results were obtained by transfection in the presence of cationic polymers.

We have tested a variety of chemical based methods for introducing exogenous DNA into human ES cells. These include cationic lipid reagents, non-liposomal formulations, and cationic polymer transfection reagents. Examples of these categories of reagent include lipofectamine (cation lipid), electroporation, FuGENE (non-liposomal formulations) and ExGen (cationic polymers including preparations of linear polymers of ethyleneimine).

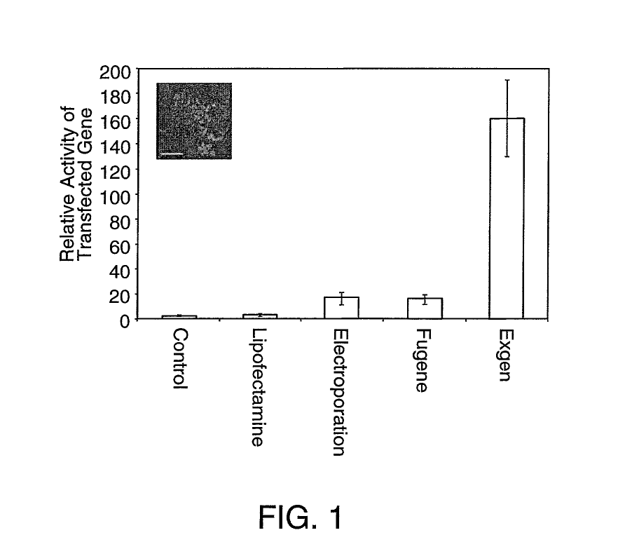

Example 1 and FIG. 1 shows the results of using Firefly Renilla Luciferase (luc) reporter gene, driven by the Herpes Simplex TK promoter (Dual Luc Reporter Assay Kit, Promega) introduced into growing colonies of human ES cells, either by LipofectAMINE™ (cationic lipid) (Life Technologies, Bethesda, Md.), FuGENE™ 6 (non-liposomal formulation) (Boehringer Mannheim, Germany) or ExGen™ 500 (cationic polymer reagent) (Fermentas, Hanover, Md.), according to the manufacturer's protocols. After 48 hours, transfection efficiencies were evaluated by measuring the relative activity of the enzyme in the presence of its substrate, in respect to protein concentration. A clear difference in transfection efficiency could be demonstrated between ExGen 500, as compared to FuGENE 6, LipofectAMINE and electroporation. Transfection with ExGen 500 delivered DNA into human ES cells with an increased efficiency of an order of magnitude over other reagents (FIG. 1).

The introduction of markers into embryonic stem cells according to the specificity of the promoter permits the separation of transfected cells from non-transfected cells or the separation of one type of transfected cell from other transfected cells. Markers can include a novel physiological feature for example a feature that enables transfected cells to grow where they could not otherwise, an example being a drug resistance gene such as Neo which confers cytoprotection to cells growing in G418. Other types of markers may facilitate the physical separation of different cell types from a mixture using biochemical analysis (protein assays, enzymatic assays, receptor binding assays) or immunological assays. Where the marker is an optical or laser recognizable feature, transfected cells expressing marker may be recognized either in vivo or in vitro according to whether the marker is expressed or not. Single or multiple marker genes may be introduced into cells. DNA encoding the markers may be associated with other genes forming fusion proteins or DNA that may specifically direct differentiation or modulate the amounts of a particular target protein produced by the differentiated cells. The expression of markers in transfected cells is regulated by promoter and enhancer sequences which result in production of amounts of protein.

We have obtained pure clones of human ES cells that are genetically modified so that their undifferentiated phenotype can be followed, and selected for in vitro. For example, a reporter gene, green fluorescent protein (EGFP) (see U.S. Pat. No. 6,316,181 for a description of green fluorescent protein marker), under the control of an ES-specific promoter was introduced into the human ES cells. By tagging the undifferentiated cells with green fluorescence, we monitored the differentiation status of the cells in culture during growth and propagation as well as following spontaneous and induced differentiation. We used the well-characterized promoter sequence of the mouse Rex-1 gene. Rex-1 is a retinoic acid-regulated zinc finger protein, which is expressed in undifferentiated ES and EC cells and in the inner cell mass (ICM) of day 4.5 mouse blastocysts (Hosler et al., (1993) Mol. Cell. Biol., Vol. 13, pp. 2919-2928, Rogers et al., (1991) Development, Vol. 113, pp. 815-824). Other promoter sequences suitable for selected expression in embryonic stem cells include Oct-4, Oct-6, SSEA-3, SSEA-4, TRA 1-60, TRA 1-81, GCTM-2, alkaline phosphatase, Hes 1 and other homeobox genes. In Example 4, Rex-1 promoter was used as a representative ES specific promoter that is rapidly down regulated upon differentiation of the embryonic cells. By introducing Rex-1-regulated selectable gene markers into human ES cells, we expressed these genes in a pluripotent-dependent fashion and determined the differentiation status of these cells in culture.

In another embodiment of the invention, a Rex-1-EGFP expression vector, which includes an SV40-Neo selectable marker, was constructed and delivered (by ExGen) into proliferating undifferentiated human ES colonies. The following day, cells were trypsinized and replated on a feeder of MitC-treated MEFNeo+, allowing the clonal propagation of transfected cells by G418 selection. By 14 days in culture, Neo resistant fluorescent labeled colonies were isolated and propagated for several passages, to allow the establishment of individual cell lines, derivatives of single transfected human ES cells (Example 1).

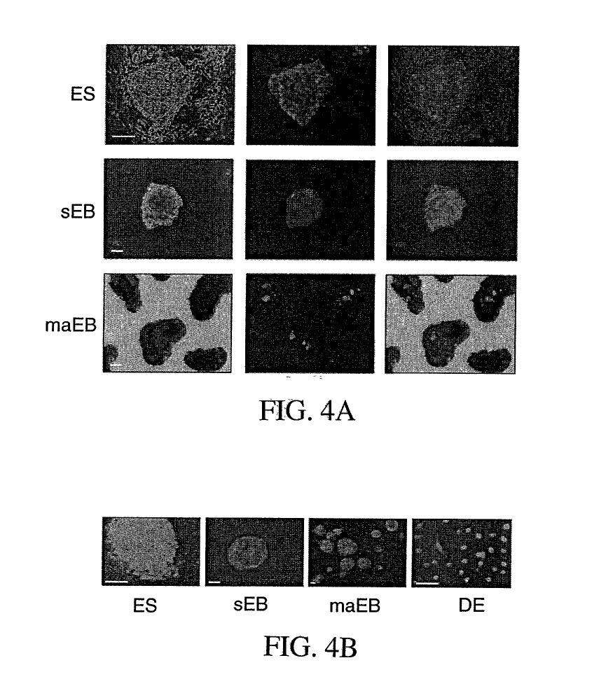

Of the 10 established cell lines, 4 were examined for pluripotency by growth under different culture conditions. When grown on feeder cells in the presence of leukaemia inhibitory factor (LIF) (to support undifferentiated growth), high expression of EGFP is detected in the small and densely packed cells of the undifferentiated colony. The fluorescent lightening overlaps well with the discrete margins of the colony and is absent in the periphery, where spontaneous differentiation is taking place (FIG. 4). By growing the cells as a monolayer in the absence of a feeder layer and LIF, we show that the labeling is highly correlated with cell morphology and does not exist in the large and loosely attached cells that grow beyond and apart from the colony. Moreover, when induced to differentiate by growing as cell aggregates in suspension culture, fluorescence gradually declines. Initially, in the outer surface of 4 day simple EBs, where a layer of primitive endodermal cells is formed, and later, if maintained to form cystic EBs (20 days in suspension), practically ceases, apart from few cores of undifferentiated cells (FIG. 4).

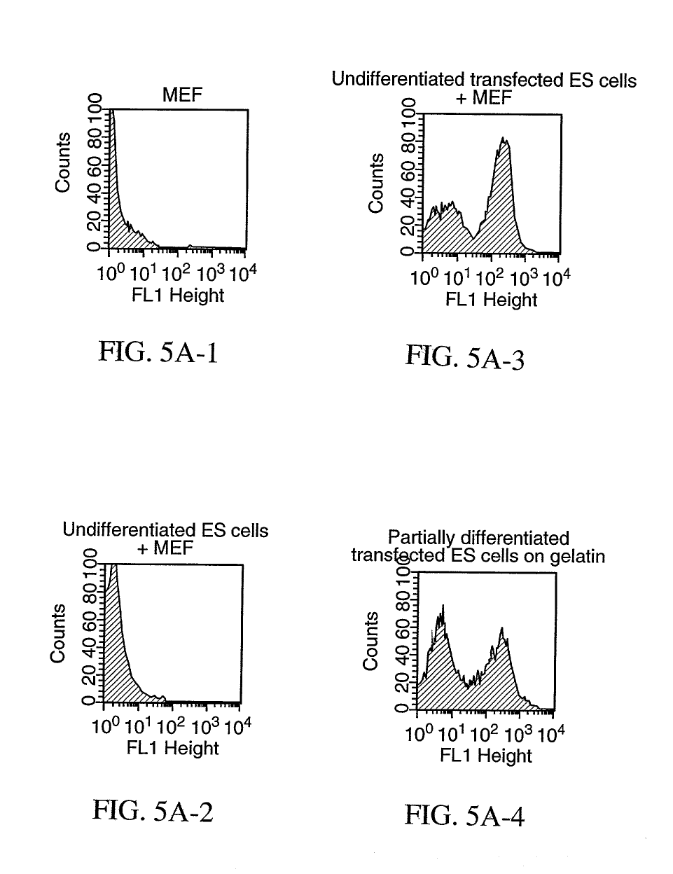

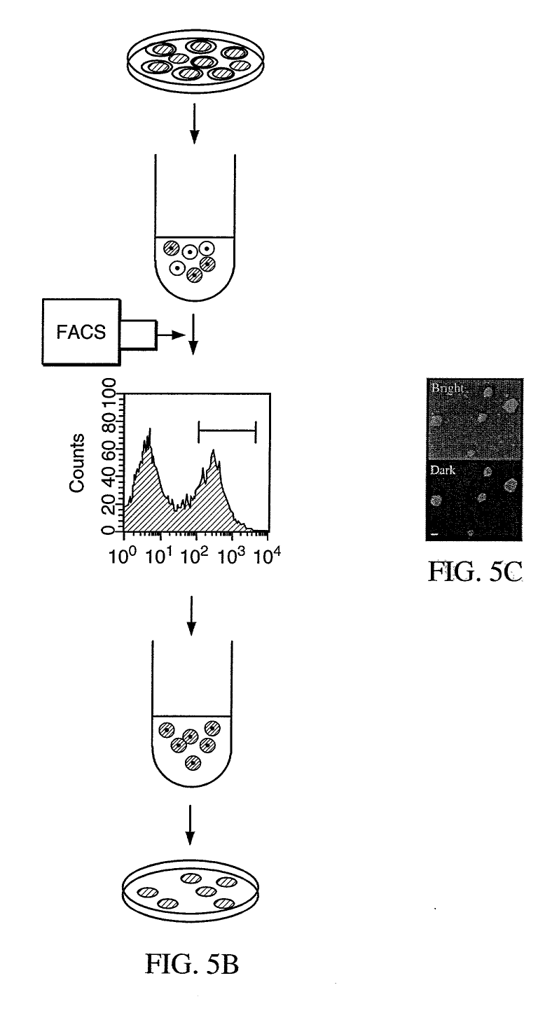

We distinguished between populations of undifferentiated and differentiated human ES using a fluorescence-activated cell sorter (FACS) (FIG. 5) or by other fluorescent activated methods such as laser scanning cytometry (Kamentsky et al., (1997) Acta Cytologica, Vol. 41, pp. 123-143) by magnetic methods (Dolan et al., (1999) U.S. Pat. No. 5,985,153) or by other methods known in the art. Cell samples of mouse embryonic fibroblasts (MEF), undifferentiated human ES cells and undifferentiated and differentiated transfected cell lines have been characterized according to their fluorescent emission. A clear difference in fluorescent intensity exists between the undifferentiated cultures of untransfected and transfected cell lines. In addition, when comparing EGFP transfected human ES to their differentiated derivatives, a shift in emission is observed (FIG. 5).

In an embodiment of the invention, we established genetically engineered cell lines of human ES, which express EGFP, under the control of mouse Rex-1 promoter. The ES-specific expression of this marker gene has allowed us to identify human pluripotent embryonic stem cells during their growth and differentiation in vitro. By growing the cells under different culture conditions we show a direct association between cell morphology and pluripotency: high EGFP expression in the densely packed cells of the undifferentiated colony is immediately turned off in the large and loosely attached cells that initiate differentiation. In addition, we demonstrate how pluripotent stem cells are rapidly eliminated in a developmentally regulated pattern, during the formation of EBs. This begins in the outer surface of simple EBs, where a layer of endodermal cells is formed, and proceeds by their elimination from most parts of the cystic EBs. These observations are similar to those in mice, where differentiation has been proven to be the cause for pluripotent stem cell extinction (Mountfort et al., (1998) Reprod. Fertil Dev., Vol. 10, pp. 527-533).

The uniform expression of Rex1-regulated sequence by the cells in the growing colony illustrates that these cells have not lost the features of the ICM during their long cultivation and are thus truly pluripotent. Moreover, since these genetically engineered cell lines have been established by the clonal expansion of single transfected cells, the possibility of variation in the developmental potential of the cells in the undifferentiated colony can be ruled out.

We have developed a stem cell selection approach in an attempt to facilitate the maintenance of human ES cells in vitro. Currently, the available methods applied for this purpose involve the identification and isolation of single colonies under a dissecting microscope; however, these procedures are time consuming and labor intensive. As an alternative we present a method for purifying undifferentiated cells, by cell sorting of fluorescent labeled cells, from mixed populations of cells. Similar selection of undifferentiated clones may be achieved by introducing into the cells a gene that enables drug selection (such as neo resistance gene) under the regulation of a human embryonic stem cell promoter such as Rex-1 or Oct-4 and maintaining the cells in the presence of the selection drug (such as the G418 antibiotic). By generating pure populations of undifferentiated cells, as described above, we can avoid the loss of human ES cultures due to their spontaneous differentiation in vitro.

Introducing a cell-specific selectable marker to the genome of undifferentiated human ES cells provides a model for isolating specific cell types for transplantation from heterogeneous cell cultures obtained by induced differentiation. In addition, the positive or negative effect of selected biological agents on directing differentiation of the stem cells to a desired cell type can be determined so as to maximize numbers of the desired cells for transplantation purposes. The ability of various biological agents to increase, decrease or modify in some way the number and nature of differentiated cells derived from embryonic stem cells may facilitate the effective use of embryonic stem cells as reagents for transplantation. In summary, the ability to transfect human embryonic stem cells with exogenous DNA containing marker genes or master genes under tissue specific promoters provides new opportunities to analyze the effect of an agent or culture environment on the outcome of differentiation of embryonic stem cells. Moreover, the assay may be used to establish the effect of an agent or culture condition to enhance or diminish an amount of a selected differentiated target cell type.

The potential use of human stem cells in transplantation and other cell based therapies require attention to the clinical problems that may arise upon residual proliferation of the cells after transplantation, causing tumor formation or ectopic and excessive differentiation. Human embryonic stem cells are a powerful tool for transplantation medicine. These cells have the ability to proliferate indefinitely in culture while remaining pluripotent. In addition, ES cells may be genetically manipulated so as to evade the host immune system. Thus they may serve as a cell source that is both unlimited and widely applicable (reviewed in Solter, (2000) Nat. Rev. Genet., Vol. 1, pp. 199-207; Pera, (2001) Curr. Opin. Genet. Dev., Vol. 11, pp. 595-599). One of the risks in the use of stem cells is their ability to massively proliferate. This may become a problem if residual amounts of stem cells remain present in the grafted sample and take on inappropriate fates (Freed, (2001) N. Engl. J. Med., Vol. 344, pp. 710-71).

The methods described herein may be used to eliminate human ES prior to or after transplantation by negative selection, so as to avoid the risk of tumor induction. In these circumstances, cells expressing a gene product from exogenous DNA are caused to die when selection is applied. In addition, clinical complications that may arise from stem cell transplantation can be avoided by transplanting cells containing genes expressing a product that may kill cells in the presence of an administered therapeutic agent. Examples of “suicide” genes include a tetracycline inducible form of the diphtheria toxin (Maxwell, (1986) Cancer Res., Vol. 46, pp. 4660-4664) and the bacterial cytosine deaminase (Pandha, (1999) J. Clin. Oncol., Vol. 17, pp. 2180-2189). Any “suicide” gene known in the prior art may be used for the negative selection of embryonic stem cells or their products in vivo or in vivo in the manner described herein. “Suicide” genes controlled by an ES specific promoter will allow elimination of the stem cell population only.

In an embodiment of the invention, we have genetically engineered human ES cell lines to constitutively express the herpes simplex thymidine kinase (HSV-TK) gene. Expression of the HSV-TK protein confers sensitivity to the FDA-approved drug Ganciclovir, allowing specific ablation of HSV-TK+ cells at concentrations non-lethal to other cell types. As expression of this gene causes sensitivity to the FDA approved pro-drug Ganciclovir (i.v. ganciclovir) it allows specific targeting of injected cells, allowing non-intrusive removal of grafts in case of unwanted side effects.

Ganciclovir (GCV), currently marketed by Roche, is an antiviral agent that interferes with DNA synthesis. The drug is phosphorylated by viral thymidine kinases, and undergoes further phosphorylation by cellular kinases. In its triphosphorylated form, the drug inhibits DNA polymerase by acting as a terminator in the synthesis of DNA, and induces apoptosis in cells.

Our results show that indeed, at a large range of Ganciclovir concentrations (10⁻⁵-10⁻⁷), which do not affect normal cells, HSV-TK+ ES and tumor cells are eliminated. Moreover, blood concentrations in patients receiving the recommended intra-venous dose of 5-50 mg/Kg (Pescovitz, (1999) Transpl. Infect. Dis. 1 Suppl., Vol. 1, pp. 31-34) should be in the range of 2×10⁻⁴ to 2×10⁻² M. Plasma measurements of Ganciclovir following a daily dose of 5 mg/Kg show that the actual steady state rate is higher than 10⁻⁶M (Klatzmann, (1998) Hum. Gene. Ther., Vol. 9, pp. 2585-2594). This concentration is in the range that effectively killed all the cells in the in-vitro experiments. As reversion rates are low it seems that in the event of malignant transformation, treatment with the pro-drug may reduce tumor mass by more than 6 orders of magnitude, allowing faster elimination of the cells. The potential of these cells to be used in transplantation medicine is enhanced by the fact that they have retained the normal properties of ES cells. This is demonstrated by the fact that no karyotype changes have occurred during the manipulation process. This is important in that it reduces the risk of malignant transformation upon injection into hosts. Perhaps more importantly, we have shown that genetically modifying the cells has not changed their capacity to differentiate into a wide variety of tissues. The expression of markers for neuronal, cardiac and pancreatic cells gives hope that they may be used in the treatment of diseases such as neurodegenerations, cardiomyopathies and diabetes mellitus. In order to ascertain the safety of selective elimination of human ES cells in-vivo, cellular transplantation experiments using animal models such as diabetic mice or spinal cord injured rats must be performed.

Previous experiments with “suicide” genes have focused on the treatment of malignancies by the use of gene therapy. Most gene therapy research has focused on introduction of the HSV-TK due to its reduced side effects (no leakage of toxin and resistance of body cells). Advanced stage clinical trials are ongoing for several malignancies such as melanomas and glioblastomas with some success (Klatzmann, 1998). One of the main problems in gene therapy is the delivery system, a problem that does not exist while working with human ES cells. These cells can be grown indefinitely in culture allowing the production of pre-made genetically modified clones at need. Our results point to the ability to make human ES cells a safer reagent in transplantation medicine without sacrificing their pluripotent nature.

All references described in this description are herein incorporated by reference.

### EXAMPLES

### Example 1

### Protocol for Transfection of Human Embryonic Stem Cells

DNA was introduced into human embryonic stem cells using electroporation, or transfection with Lipofectamine plus (Invitrogen Life Technologies, Gruningen, The Netherlands), with FuGENE (BoehringerMannheim Manheim, Germany) and with ExGen (Fermentas, Hanover, Md.). An example protocol is provided on page 25.

Cell Culture: Human ES cells were grown on a feeder layer of mouse embryonic fibroblasts (MEF) and then transferred to gelatin coated plates and cultured further to reduce the number of murine cells in the culture. Differentiation into embryoid bodies (EBs) was initiated by transfer to petri dishes, where the embryoid bodies remained in suspension. (Schuldiner 2000) differentiated embryonic (DE) cells were formed by dissociating the EBs after 5 days and culturing them as a monolayer.

More specifically, human ES cells were obtained as described in Thomson et al., (1998) Science Vol. 286, pp. 1145-1147. Cleavage stage human embryos produced by in vitro fertilization (IVF) were obtained after the requisite approval process. The embryos were cultured to the blastocyst stage and inner cell masses were isolated. ES cell lines were isolated from the embryos and a cell line was selected which had a normal XX karyotype after 6 months of culture and could be passaged continuously over several months without undergoing a period of replicative crisis. The cells had a uniform undifferentiated morphology when grown on Mitomycin-C treated mouse embryonic fibroblast feeder layer (obtained from day 13.5 embryos) in 80% KnockOutTM DMEM medium (Gibco-BRL), supplemented with 20% KnockOutTM SR - serum-free formulation (Gibco-BRL), 1 mM glutamine (Gibco-BRL), 0.1 mM βmercaptoethanol (Sigma), 1% non-essential amino acids stock (Gibco-BRL), Penicillin (50 units/ml) and Streptomycin (50 μg/ml), in the presence of 103 units/ml of leukemia inhibitor factor (LIF) (Gibco-BRL), and 4 ng/ml of basic fibroblast growth factor (bFGF) (Gibco-BRL) (Schuldiner et al., 2000).

The undifferentiated cell cultures were induced to differentiate in vitro into EBs by omitting LIF and bFGF from the growth media and allowing aggregation in petri dishes (Schuldiner et al., 2000). Alternatively, undifferentiated cells were allowed to undergo spontaneous differentiation by growing as a monolayer on 0.1% gelatin (Merck) coated plates, in the absence of LIF and bFGF.

Teratomas were formed by injecting the cells into nude mice (Robertson, (1987), tumors were removed, dissociated and the cells grown on 0.1% gelatin coated plates with human ES cells media. Ganciclovir (Sigma G2536) was administered one day after plating at the stated concentrations and media was changed with fresh Ganciclovir every two days.

Plasmid Construction: A Rex-1-EGFP expression vector was constructed by the deletion of the CMV promoter sequence from pEGFP-N1 (Clontech), and introduction of the mouse Rex-1 promoter sequence (700 bp) intro a HindIII restriction site.

Transient transfection: To determine the efficiency of DNA transfection by each of the above methods, cells were transfected with a construct of firefly Rennila protein under the control of the TK promoter. The cells were harvested 48 hours following transfection and luminosity of the Rennila protein was monitored using a luminometer. Results are provided in the histogram of FIG. 1 showing the relative activity of the transfected gene (luminosity units per mg protein). Each experiment was repeated three times and standard error is given in each experiment. As can be seen from FIG. 1, the most efficient method for transfection of hES occurred when Exgen reagent was used.

Transfection and Establishment of Transgenic Cell Lines: Fully expanded and undifferentiated human ES cells underwent stable transfection with Rex-1-EGFP plasmid DNA (Rex-1 is a gene specific to undifferentiated ES cells (the Rex-1 gene) ExGen-500 transfection system (Fermentas) was used. Transfection was carried out in the human ES cells in 6-well plates on feeder cells, and was performed as described by the manufacturer's protocol. Specifically, the cells were incubation at room temperature with the transfecting agent for 10 minutes (2 μg of plasmid DNA plus 1 μl of ExGen 500 in 1 ml media per well), centrifugation at 1100 rpm for 5 minutes and incubation at 37° C. in a moist chamber for 45 minutes. Residuals of transfecting agent were removed by washing twice with PBS. The following day, the cells were trypsinized and re-plated on 10 cm² culture dishes containing inactivated MEFNeo+. Two days after transfection G418 (200 ng/ml) was administered to the growth medium, allowing the selective propagation of transfected cells in culture. By day 14, Neo resistant fluorescent-labeled colonies were identified by a fluorescent microscope. Single transgenic colonies were picked off by a micropipette, dissociated into small clumps of cells and transferred into a 2 cm² (24-well) culture dish, on a fresh feeder of MEFNeo+. The cells continuously proliferated in the presence of G418 and formed a large number of expanding undifferentiated colonies, which express EGFP consistently high by all of the cells within the colony. Overcrowded cultures were trypsinized and propagated in 10 cm² culture dishes for several passages to allow the establishment of individual cell lines, derivatives of a single transfected human ES cells.

Shown in FIG. 4 are the transfected ES cells and their differentiation as a simple embryoid body (sEB), or as cystic embryoid bodies (cEB). In FIG. 4, the left and middle rows are photos of light and dark fields respectively. The right row is the overlay of the two photos. Note that only the undifferentiated cells are fluorescent. The fluorescent ES clone is surrounded by differentiated non-fluorescent cells. The simple EB is labeled only in the middle and not in the peripheral primitive endodermal cells. Cystic EBs are generally not fluorescent and only very distinct area in them are still fluorescent (probably residual undifferentiated cells.)

FACS Analysis FACS analysis of Rex1-EGFP expressing cells was performed on a FACSCalibur system (Becton-Dickinson, San Jose, Calif.), according to cell size, granularity and fluorescent intensity. Undifferentiated human ES cells were used to set the background level of fluorescence. Transfected cells of undifferentiated (grown on MEF in the presence of LIF) and partially-differentiated (obtained by growth on gelatin in the absence of LIF) cell cultures were analyzed for fluorescence intensity as compared to background control.

### Example 2

### Transfection with DNA for Directed Differentiation

Transcription factors that regulate differentiation of specific cells (master genes) are transfected into human embryonic stem cells according to the method described in Example 1. The expression of the transfected master gene should allow further differentiation of human ES cells in a regulated manner with a predetermined outcome.

### Example 3

### Transfection of Embryonic Stem Cells with DNA Encoding a Cell Specific Marker

Embryonic stem cells were transfected with a DNA encoding a cell specific marker linked to marker genes according to Example 1. Cells expressing the marker can be monitored in culture, and selected for or sorted by fluorescent activated cell sorting (FACS) to provide a purified preparation of a particular type of cell. Such a system allows analysis of cells such as neuronal cells (when using a neuronal specific enhancer such as the enhancer for neurofilament light chain gene); heart muscle cells (when using a cardiomyocyte specific enhancer such as the enhancer for alpha cardiac actin gene); liver cells (when using a hepatocyte specific enhancer such as the enhancer for albumin gene); or pancreatic cell (when using a pancreatic islet specific enhancer such as the enhancer for the insulin gene).

### Example 4

### Transfection of a Human Embryonic Stem Cells With a Fluorescent Protein to Identify Undifferentiated Cells

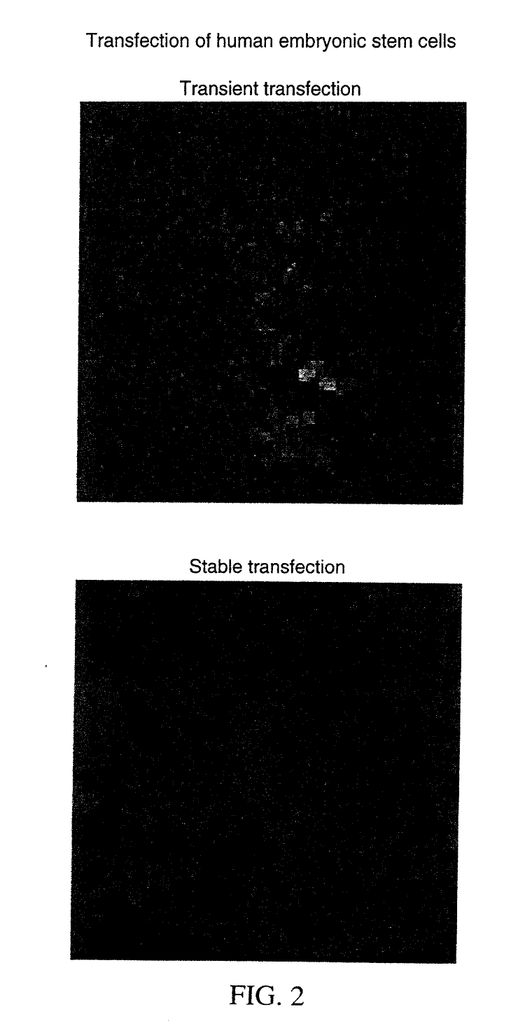

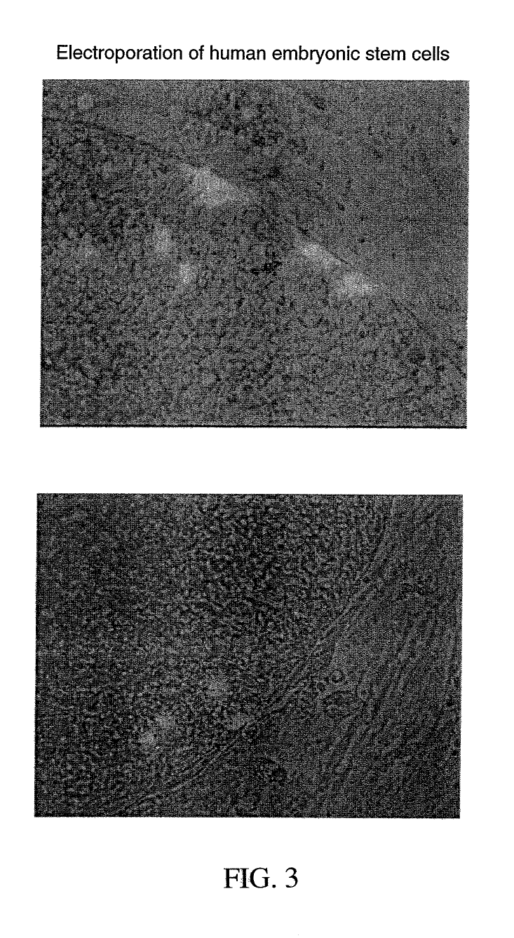

Embryonic stem cells were transfected with a DNA containing an enhancer specific to undifferentiated cells (such as the enhancer for Rex-1 or Oct-4) linked to marker genes, according to Example 1. Cells expressing the marker can be followed in culture, selected for or sorted by fluorescent activated cell sorting (FACS) to provide a purified preparation of undifferentiated cells (see FIG. 2 for a colony of cells containing a fluorescent protein under an enhancer specific to undifferentiated cells).

### Example 5

### Creation of Genetically Modified Clones

Cell culture—Human ES cells (Thomson, 1998) and EBs were cultured as described (Schuldiner, 2000). Teratomas were formed by injecting ES cells into nude mice (Robertson, 1987), tumors were removed, dissociated and the cells grown on 0.1% gelatin coated plates with human ES cells media. Ganciclovir (Sigma G2536) was administered one day after plating at the stated concentrations and media was changed with fresh Ganciclovir every two days. Cell densities were followed by staining with Methylene Blue (Sigma) dissolved in 0.1M Boric Acid (pH=8.5) after fixation with 2.5% Glutaraldehyde. Color was extracted by 0.1N HCl and emission was read at 650 nM.

Transfection and establishment of transgenic cell lines Plasmids introduced to the human ES cells were a PGK-EGFP plasmid (example 1) or a PNT plasmid (Tybulewicz et al., (1991) Cell, Vol. 65, pp. 1153-1163) that contains two PGK promoters driving either neomycin resistance gene or the herpes simplex thymidine kinase gene. Transfection was performed using ExGen 500 (Fermentas) as described in Example 1. FACS analysis and cell sorting—FACS analysis of PGK-EGFP and PNT expressing cells was performed on a FACSCalibur system (Becton-Dickinson, San Jose, Calif.), according to their green fluorescent emission.

RNA and RT-PCR—Total RNA was extracted as described (Chomczynski, P. and Sacchi, N. (1987) Anal. Biochem. 162, 156-159) and cDNA was synthesized from 1 μg total RNA, using random hexamer (pd(N)6) as primer (Pharmacia Biotech) and M-MLV reverse transcriptase (Gibco-BRL). cDNA samples were subjected to PCR amplification with previously described DNA primers (Schuldiner, 2000) and primers for GFAP: SEQ ID. NO. 1: cgagaacaacctggctgcctatag and SEQ. ID NO. 2: gtgggtcctgcctcacatcacatc.

Cytogenetic analysis: Cells prepared for cytogenetic analysis were incubated in growth media with 0.1 μg/ml of Colcemid for 10 min, trypsinized, re-suspended in 0.075M KCL and incubated for 10 min at room temperature, then fixed in 3:1 methanol/acetic acid and G-banded.

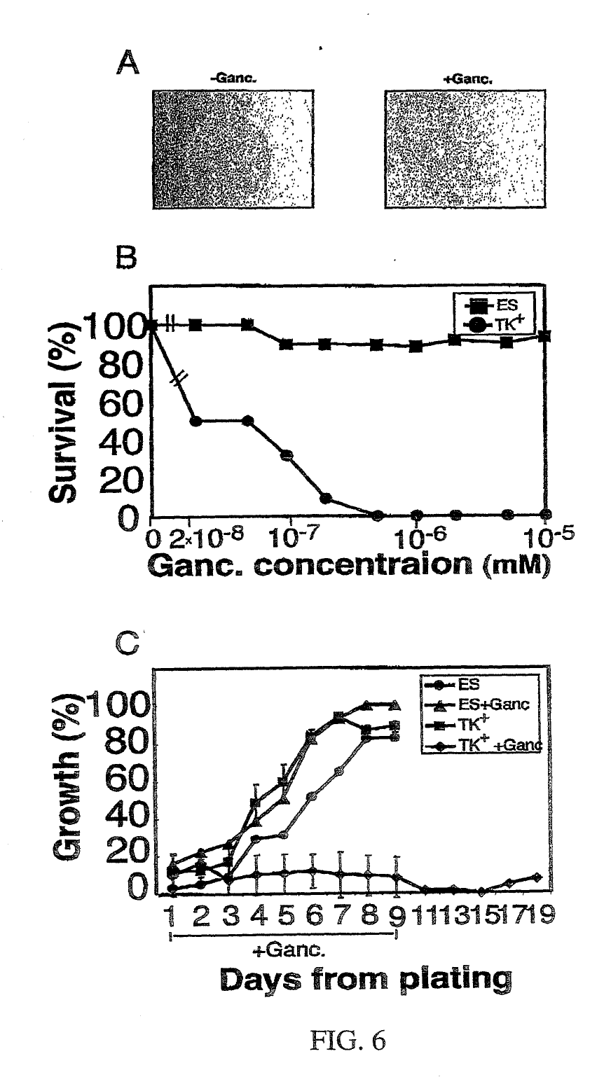

In order to create lines of cells carrying a negative selection marker we transfected human ES cells with a plasmid encoding the thymidine kinase gene from herpes simplex (HSV-TK) under control of a house-keeping gene (phosphoglycerate kinase-PGK) promoter. The introduced plasmid vector also contains the neomycin resistance gene that allowed selection for cells harboring the foreign DNA. Expression of the HSV-TK causes conversion of the pro-drug nucleoside Ganciclovir to its drug form as a phosphorylated base analog. The phosphorylated Ganciclovir is incorporated into the DNA of replicating cells causing irreversible arrest at the G2/M checkpoint followed by apoptosis (Halloran, (1998) Cancer Res., Vol. 58, pp. 3855-3865; Rubsam, (1998) Cancer Res., Vol. 58, pp. 3873-3882). Due to the high specificity of the cellular TK, Ganciclovir is not converted to the active form in normal eukaryotic cells causing insensitivity to its possible deleterious effects. All isolated lines of human ES cells transfected with HSV-TK were terminally arrested in response to the administration of Ganciclovir (FIG. 6a). The transfected clones died in the presence of a range of Ganciclovir concentrations varying over three orders of magnitude (from 2×10⁻⁸M to 1×10⁻⁵M), while the control human ES cells remained unaffected (FIG. 6b). We chose to continue working at an intermediate concentration of 2×10⁻⁶M Ganciclovir, in which all HSV-TK+ cells died. At this concentration removal of Ganciclovir from the media after nine days of selection did not result in renewed growth of the cells, indicating that the action of the drug was terminal (FIG. 6c).

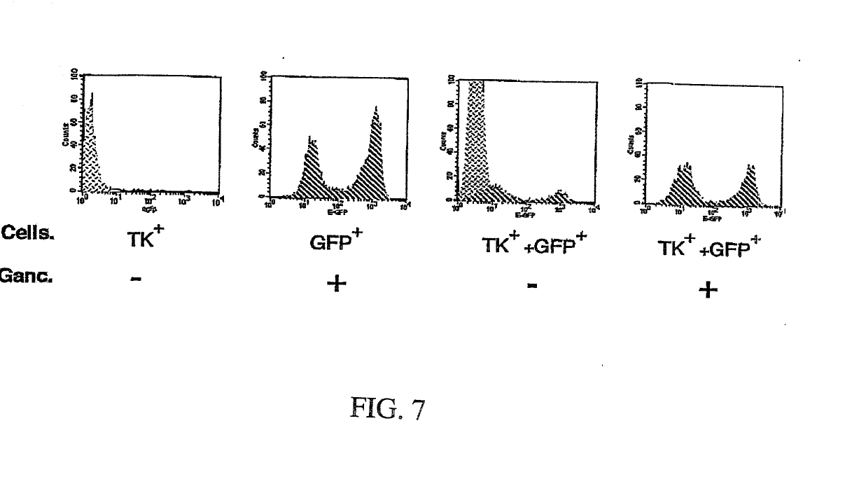

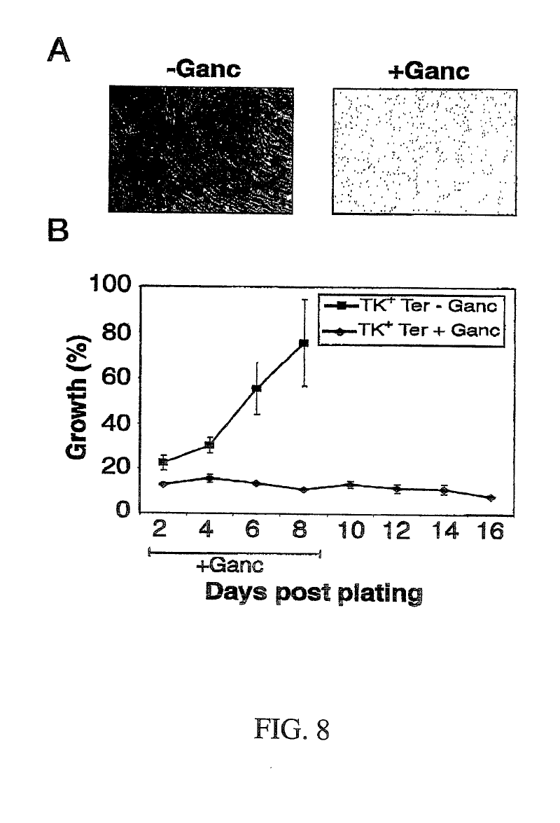

Susceptibility of HSV-TK+ cells to Ganciclovir: Transplantation of human ES cells will expose them to conditions differing than those encountered in culture. First, they will differentiate over the passage of time and will also be surrounded by cells that are not affected by the treatment. Moreover, in vivo they will be free from the positive selective pressure of the neomycine to retain the plasmid and may be under strong negative selective pressure in favor of those cells that have lost the HSV-TK gene if treatment with Ganciclovir commences. These problems may cause resistance to the treatment suggested. In order to examine whether HSV-TK+ cells can be selectively eliminated, we have grown these cells together with cells expressing the green fluorescent protein (GFP+) and treated them with Ganciclovir. When the cultures were analyzed using a fluorescence activated cell sorter (FACS), it was clear that selective killing of the HSV-TK+ cells occurred at the same time scale and dose as with cultures containing HSV-TK+ cells alone (FIG. 7). Analysis of the effects of differentiation was performed by taking differentiated teratoma cells that have formed following injection of the clones into immunodeficient mice. The teratomas were removed from the mice after 8 weeks in which they were not subject to selection and the cells that were grown in culture retained their susceptibility to Ganciclovir at the same concentrations previously used (FIG. 8a, b). This analysis also confirms that the fetal cells that are not grown continuously on neomycin selection still retain HSV-TK expression. In addition, we monitored the reversion rate of the Ganciclovir susceptibility in order to get an estimation of the possible number of drug resistant cells that arise following injection. The cells were grown for two passages in the absence of the positive selection drug neomycin, and then re-plated in the presence of Ganciclovir and grown for ten additional days and resistant colonies counted. Reversion was noted at a frequency between 10⁻⁶-10⁻⁷.

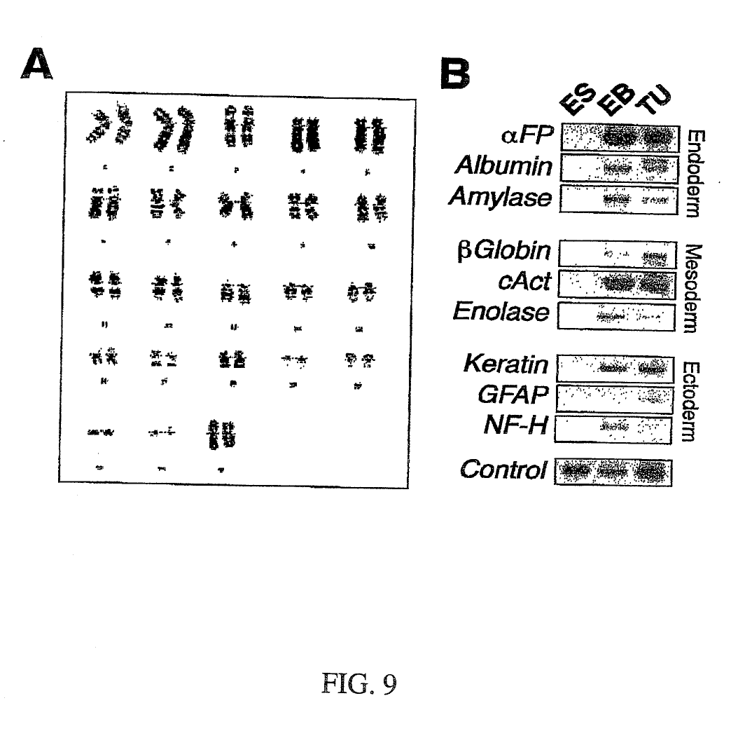

Retention of nornal karyotype and differentiation potential of modified clones: Our aim was to ascertain that the genetically modified clones still retained their normal, pluripotent nature. Cytogenetic analysis of the transfected clones revealed a normal karyotype (FIG. 9a). In addition, the cells could easily aggregate to form normal looking embryoid bodies (EBs) (Itskovitz-Eldor, 2000) in vitro and teratomas in vivo. RT-PCR analysis of cDNA from both EBs and Teratomas cells revealed a capacity to differentiate to cells from all three germ layers and to a wide variety of cell types as could be seen by the expression of the endodermal tissue specific markers: α-fetoprotein (primitive endoderm) albumin (liver) and amylase (pancreas); the mesodermal markers: β globin (blood), c-actin (cardiac muscle) and enolase (muscle); and the ectodermal markers: keratinl (skin), GFAP-glial fibrillary acidic protein (Glia) and neurofilament (mature neurons) (FIG. 9b).

### Example 6

### Immunological Tolerance in Transplanted Cells

We target the genes of the major histocompatibility complex in embryonic stem cells and their differentiated progeny. This is achieved by knock-out or inhibition (by anti-sense or dominant negative form overexpression or ribozymes) of beta2 microglobulin or HLA-1 or HLA-11 or INF receptors. Any known method for inserting, deleting or modifying a desired gene from a mammalian cell combined with the transfection techniques described in Examples 1-5 can be employed. Methods and vectors for effecting gene knockout are the subject of numerous patents including U.S. Pat. Nos. 6,074,853, 5,998,144, 5,948,653, 5,945,339, 5,925,544, 5,869,718, 5,830,698, 5,780,296, 5,614,396, 5,612,205, 5,468,629, 5,093,257 all of which are incorporated by reference in their entirety herein.

What is claimed is:

1. A method of altering gene expression in a population of human embryonic stem cells; comprising:
introducing a polynucleotide into the population of cells, the polynucleotide containing a gene expression altering sequence so that gene expression in the embryonic stem cells prior to introducing the polynucleotide is measurably different from gene expression after introducing the polynucleotide while retaining the pluripotent character of the cells.

2. A method according to claim 1, wherein the expression altering sequence is an enhancer for modulating gene expression in the population of embryonic stem cells.

3. A method according to claim 1, wherein the expression altering sequence is a gene encoding a protein, the protein not expressed in the population of embryonic stem cells absent the polynucleotide.

4. A method according to claim 3, wherein the protein is selected from a fluorescent protein and an antibiotic resistance protein.

5. A method according to claim 4, wherein the fluorescent protein is selected from green fluorescent protein, lacZ, firefly Rennila protein, luciferase, red cyan protein and yellow cyan protein.

6. A method according to claim 4, wherein the antibiotic resistance protein is selected from hygromycin, neomycin, zeocin and puromycin.

7. A method according to claim 1, wherein the polynucleotide is formulated with a cationic non-lipid polymer transfection reagent for introducing the polynucleotide into the population of cells.

8. A method according to claim 1, wherein the polynucleotide is formulated with a liposomal transfection reagent for introducing the polynucleotide into the population of cells.

9. A method according to claim 1, wherein the polynucleotide is formulated with a cationic lipid reagent for introducing the polynucleotide into the population of cells.

10. A method according to claim 1, wherein the polynucleotide is introduced by electroporation into the population of cells.

11. A method of altering gene expression in a population of human embryonic stem cells; comprising:
introducing into the population of cells by electroporation or in the presence of a cationic polymer, a DNA sequence corresponding to at least one of an enhancer, a promoter and a gene so as to alter gene expression in the population of embryonic cells in an amount to permit cells containing the DNA sequence to be distinguished from cells absent the DNA sequence.

12. A method according to claim 11, wherein the DNA sequence corresponds to a gene and the gene encodes a protein selected from a fluorescent protein, a suicide gene, and an antibiotic resistance protein.

13. A method according to claim 11, wherein the DNA sequence corresponds to a promoter and the promoter is selected from rex-1, oct-4, oct-6, SSEA-3, SSEA-4, TRA1-60, TR1-81, GCTM-2, alkaline phosphatase, and Hes1 promoters.

14. A method according to claim 12, wherein the fluorescent protein is selected from green fluorescent protein, lacZ, firefly Rennila protein, luciferase, red cyan protein and yellow cyan protein.

15. A method according to claim 12, wherein the protein is an antibiotic resistance protein and the antibiotic resistance protein is selected from hygromycin, neomycin, zeocin and puromycin.

16. A method according to claim 12, wherein the DNA corresponds to a suicide gene and the suicide gene is an inducible apoptic gene or encodes a protein selected from herpes simplex thymidine kinase, inducible Diptheria toxin, bacterial cytosine deaminase.

17. A method according to claim 11, wherein DNA sequence causes a knockout of a genomic sequence the genomic sequence selected from beta 2 microglobulin, HLA-1, HLA-2 or an INF receptor gene sequences.

18. A method for purifying pluripotent embryonic stem cells from a heterogeneous population of cells, comprising:
(a) introducing into the cells, a DNA encoding a selectable marker under a promoter that is specifically active in undifferentiated cell;
(b) separating those cells expressing the selectable marker from cells not expressing the marker; and
(c) obtaining purified pluripotent cells.

19. A method according to claim 18, wherein the selectable marker is a fluorescent marker.

20. A method according to claim 18, wherein the fluorescent marker is selected from green fluorescent protein, lacZ, firefly Rennila protein, luciferase, red cyan protein and yellow cyan protein.

21. A method according to claim 18, wherein (b) further comprises separating the cells containing the marker from the cells lacking the marker using a fluorescent activated cell sorter or a laser scanning cytometer.

22. A method according to claim 18, wherein the selectable marker is an antibiotic resistance marker.

23. A method according to claim 22, wherein step (b) further comprises separating cells by culturing the cells in a selective medium containing antibiotic.

24. A method for treating a human subject for a condition resulting from a deficiency of a selected cell type, comprising:
(a) causing human embryonic stem cells to be transfected in vitro with a nucleic acid encoding a marker under a tissue specific promoter;
(b) separating the selected cell type expressing the marker from cells not expressing the marker, and
(c) introducing the selected cell type into the subject so as to treat the condition.

25. A method according to claim 24, wherein the nucleic acid further contains a suicide gene.

26. A method according to claim 25, wherein the suicide gene is an inducible apoptic gene or encodes a protein selected from herpes simplex thymidine kinase, inducible Diptheria toxin, and bacterial cytosine deaminase.

27. A method according to claim 24, wherein the marker is selected from a fluorescent marker and an antibiotic resistance protein.

28. A method according to claim 27, wherein the fluorescent protein is selected from green fluorescent protein, lacZ, firefly Rennila protein, luciferase, red cyan protein and yellow cyan protein.

29. A method according to claim 27, wherein the antibiotic resistance protein is selected from hygromycin, neomycin, zeocin and puromycin.

30. A method according to claim 24, wherein the cells are transfected by means of a cationic polymer transfection reagent.

31. A method according to claim 24, wherein the nucleic acid does not contain viral genes.

32. A method according to claim 24, wherein the cell type is selected from epidermal cells, dermal cells, muscle cells, cartilage cells, osteoblasts, osteoclasts, neurons, retinal cells, endodermal cells, hematopoietic cells, cells of the immune system.

33. A method according to claim 24, wherein the cell type is selected from specialized cells from any functionally distinct organ in the human subject.

34. A method according to claim 24, wherein introducing the cell type to the subject is achieved by injection.

35. A method according to claim 24, wherein the condition is selected from cancer, an immune disorder, an autoimmune disease, a disease of aging, a degenerative disease including a neurodegenerative disease, and trauma.

36. A cell population; comprising a substantially pure population of human embryonic stem cells containing an expression altering sequence of exogenous DNA.

37. A method of producing a clonal pluripotent cell population from a mixture of pluripotent and differentiated cells; comprising:
(a) transfecting the mixture of cells in the presence of a cationic polymer or by electroporation with a DNA encoding a marker protein under a promoter that is selectively active in cells of the inner cell mass of the embryo; and
(b) separating the embryonic stem cells from the differentiated cells according to the presence or absence of an expressed marker so as to produce the clonal pluripotent cell population.

38. A method according to claim 37, wherein the promoter selected from rex-1, oct-4, oct-6, SSEA-3, SSEA-4, TRA1-60, TR1-81, GCTM-2, alkaline phosphatase, and Hes1 promoters.

39. A method according to claim 37, wherein the cell population is transfected with a DNA in the presence of a cationic polymer.

40. A method according to claim 37, wherein the marker protein is selected from a fluorescent protein and an antibiotic resistance protein.

41. A method according to claim 37, wherein the fluorescent protein is selected from green fluorescent protein, lacZ, firefly Rennila protein, luciferase, red cyan protein and yellow cyan protein.

42. A method according to claim 37, wherein the antibiotic resistance protein is selected from hygromycin, neomycin, zeocin and puromycin.

43. A method of regulating cell viability of a population of cells in a subject, wherein the population of cells are derived from a human embryonic stem cell culture which has undergone directed differentiation, comprising:
(a) introducing the population of cells into the subject, the population of cells containing an exogenous DNA encoding a suicide gene, wherein the population of cells are selected from the group consisting of: undifferentiated cells, partially differentiated or wholly differentiated cells; and
(b) treating the subject with an agent for activating a sequence of events leading to suicide in the cells in the subject in response to an adverse event associated with the introduced cells.

44. A method according to claim 43, wherein the occurrence of adverse events is a hyperproliferation of the introduced cells.

45. A method for screening an agent to determine an effect on differentiation of pluripotent cells in vitro, comprising:
(a) adding the agent to an in vitro cell culture of a population of genetically engineered human embryonic stem cells expressing a detectable marker under a cell specific promoter; and
(b) providing the conditions for the embryonic stem cells to differentiate; and
(c) determining the effect of the agent on differentiation of pluripotent cells.

46. A method according to claim 45, wherein the detectable marker is a fluorescent marker.

47. A method according to claim 46, wherein the fluorescent marker is enhanced green fluorescent protein.

48. A reagent cell population for supplying material for transplantation consisting essentially of pluripotent human embryonic stem cells modified by foreign genetic material which is DNA not normally present in embryonic stem cells; which occurs in embryonic stem cells but is not expressed in them at levels which are biologically significant; DNA which occurs in embryonic stem cells and has been modified so that it is only expressed by selected derivative cells; or any DNA that can be modified to be expressed by embryonic cells, derivative cells alone or in any combination thereof.

49. A reagent cell population according to claim 48, in which the foreign genetic material comprises genetic material encoding at least one selectable marker.

50. A reagent cell population according to claim 49, in which at least one selectable marker is a dominant selectable marker.

51. A reagent cell population according to claim 50, in which the dominant selectable marker is a gene encoding antibiotic resistance.

52. A reagent cell population according to claim 49, in which the dominant selectable marker is a gene encoding a suicide protein.

53. A reagent cell population according to claim 50, in which the dominant selectable marker is a gene encoding a fluorescent protein or an antibiotic resistant protein.

54. A reagent cell population according to claim 52, in which the gene is hsv-tk and the suicide protein is thymidine kinase or the suicide gene is an inducible apoptic gene or encodes a protein selected from inducible Diptheria toxin, bacterial cytosine deaminase

55. A reagent cell population according to claim 53, wherein the gene encodes a fluorescent protein selected from green fluorescent protein, lacZ, firefly Rennila protein, luciferase, red cyan protein and yellow cyan protein.

56. A method according to claim 53, wherein the antibiotic resistance protein is selected from hygromycin, neomycin, zeocin and puromycin


SEQUENCE LISTING (2 sequences)

SEQ ID NO: 1 - DNA, 24 bp, GFAP primer
cgagaacaacctggctgcctatag

SEQ ID NO: 2 - DNA, 24 bp, GFAP primer
gtgggtcctgcctcacatcacatc